(19) United States
(12) Patent Application Publication
(10) Pub. No.: US 2024/0285699 A1
(43) Pub. Date: Aug. 29, 2024

(54) Method to Control Administration of Active Substance to the Digestive Tract

(71) Applicant: Lonza Greenwood LLC, Greenwood, SC (US)

(72) Inventors: Aouatef Bellamine, Greenwood, SC (US); Tyler White, Greenwood, SC (US); Shane Durkee, Greenwood, SC (US)

(21) Appl. No.: 18/572,983

(22) PCT Filed: Jun. 23, 2022
(86) PCT No.: PCT/US2022/034683

§ 371 Date: Dec. 21, 2023

Related U.S. Application Data
(60) Provisional application No. 63/214,438, filed on Jun. 24, 2021.

(51) Int. Cl. A61K 35/747; A61K 9/48; A61K 35/00; A61P 1/14

(52) U.S. Cl. CPC A61K 35/747; A61K 9/4808; A61K 9/4816; A61K 9/485; A61K 9/4858; A61K 9/4875; A61P 1/14; A61K 2035/115

(57) ABSTRACT

The present disclosure is directed to method providing effective oral administration of active substance, including pharmaceutical ingredients, nutraceuticals, enzymes or probiotics using a delivery system for optimal bioactivity and absorption of the active substance by the mammal to which the active substance is delivered.

### RELATED APPLICATIONS

The present application is based upon and claims priority to U.S. Provisional Patent Application Ser. No. 63/214,438, having a filing date of Jun. 24, 2021, which is incorporated herein by reference.

### BACKGROUND

Oral administration of active substances, such as pharmaceutical ingredients, nutraceutical ingredients or probiotics is generally the preferred method to administer active substances to a mammal due to its convenience, potential controlled release, and user compliance. Despite these advantages, many challenges are associated with oral administration of active substances such as product performance, sufficient and efficient dosing of the active ingredients and the survivability of the active substance in the gastrointestinal tract active.

In the upper gastrointestinal tract (GIT), orally administered pharmaceutical ingredients, nutraceutical ingredients and probiotics are prone to degradation because of the harsh acidic conditions in the stomach and the gastric enzymes (i.e., pepsin). In the duodenum, pancreatic enzymes (i.e., lipase, trypsin, amylase, peptidases) and bile salts can significantly affect the stability of these ingredients, particularly probiotic viability. During the fasted or fed conditions, different transit times, pH profiles, and enzymatic levels have been described, requiring adjustments of oral entity dosage forms for better efficacy and performance.

Therefore, immediate-release formulations should be avoided when pH-sensitive active substances are delivered orally. For example, probiotics which are live microorganisms, confer a health benefit on the host only when administered in adequate levels and may have lower performance when the strain viability is reduced during the GIT transit because of a low pH for example. Nutritional supplements, like flavonoids, carotenoids, hydroxycinnamoyl acid or vitamin C, can also be highly degraded (80-91%) during gastrointestinal digestion, while bioactives like proteins and peptides can be damaged by the action of pepsin and trypsin degradation, thus significantly reducing their activity.

Different strategies, including tablet coating or bioactive encapsulation, have been developed to provide an adequate formulation for acid-sensitive products. Tablets have the disadvantages of low compressibility ingredients, slow dissolution or bitter taste. In addition, during the early stages of drug development, the limited amount of drug availability can impede the development of a coated pellet or tablet formulation. Therefore, certain capsule polymers, like cellulose derivatives or acrylic/methacrylic acid derivatives may offer a better solid dosage form and also provide the possibility to target the delivery of liquids or semi-solid formulations to the small or large intestine. Thus, capsule technology has made huge progress in the last years, offering economically convenient alternatives for pharmaceutical, nutraceutical and probiotic formulations, as well as functionality for targeted entity release.

### SUMMARY

In general, the present disclosure is directed to method providing effective oral administration of active substances, including pharmaceutical ingredients, nutraceuticals, enzymes or probiotics, using a delivery system for optimal bioactivity and absorption of the active substance by the mammal to which the active substance is delivered.

In a first embodiment, the present disclosure is directed to method of providing effective oral administration of an active substance to a mammal, where the active substance is delivered to the digestive tract of the mammal. The method includes preparing a delivery system where the delivery system comprises an outer capsule having an outer shell wall and an internal chamber, an inner capsule having an exterior shell wall and an inner compartment. The inner capsule being located in the internal chamber of the outer capsule, said inner capsule being acid resistant, and containing an active substance. The active substance is present in the inner compartment of the inner capsule. The delivery system is orally administered to a mammal, and the delivery system delivers the active substance in an effective amount to the intestine of the mammal.

In a second embodiment, the present disclosure is directed to method of modifying the microbiome and colonization of the gut by administering an active ingredient to the gut. The method comprises preparing a delivery system. The delivery system comprises an outer capsule having an outer shell wall and an internal chamber, an inner capsule having an exterior shell wall and an inner compartment. The inner capsule is located in the internal chamber of the outer capsule and the inner capsule can be formulated to be acid resistant. A probiotic active ingredient is present in the inner compartment of the inner capsule. The delivery system is orally administered to a mammal, and the delivery system delivers the probiotic active substance in an effective amount to the gut of the mammal. The active ingredient improves the microbiome or colonization of healthy bacteria in the gut.

In a further aspect of the present disclosure, in the delivery system, the outer capsule comprises an HPMC hard capsule.

In a further aspect of the present disclosure, in the delivery system, the inner capsule comprises a HPMC hard capsules with acid resistance.

In another aspect of the present disclosure, the inner capsule comprises a capsule comprising HPMC and gellan gum. In one particular aspect, the gellan gum is present in an amount between about 4 parts-15 parts per 100 parts of the HPMC.

In a different aspect of the present disclosure, the HPMC outer capsule comprises a thermogelled HPMC.

In a further embodiment of the present disclosure, the outer capsule is an acid resistant capsule. In one aspect, the outer acid resistant capsule is a HPMC hard capsules with acid resistance. In a particular embodiment, the outer capsule is a capsule comprising HPMC and gellan gum. The gellan gum is present in an amount between about 4 parts-15 parts per 100 parts of the HPMC.

In one embodiment, the active substance comprises a probiotic.

In a different embodiment of the present disclosure, the inner capsule and the outer capsule are each comprise acid resistant capsules and each acid resistant capsule comprises HPMC and gellan gum.

In another embodiment, the method provides a way to deliver the active substance to the colon in an amount which is at least 10 times greater than a capsule which dissolves in the stomach or small intestine. In further embodiment, the active substance is delivered to the colon in an amount which is at least 20 times greater than a capsule which dissolves in the stomach or small intestine, and even at least 30 times greater than a capsule which dissolves in the stomach or small intestine.

Other features and aspects of the present disclosure are discussed in greater detail below.

### BRIEF DESCRIPTION OF THE FIGURES

FIG. 1 shows a delivery system useable in the method of the present disclosure with capsule-in-capsule configuration.

FIG. 2 shows a delivery system useable in the method of the present disclosure with triple capsule configuration.

FIG. 3 shows a delivery system useable in the method of the present disclosure with a multiple capsule configuration.

FIG. 4 shows the pH-profile used in the Example under fed (A) and fasted (B) conditions.

FIG. 5 shows the effect of capsule configuration on caffeine release during the stomach and small intestine in simulated under fasting conditions.

FIG. 6 shows the effect of capsule configuration on caffeine release during the stomach and small intestine in simulated digestion under fed conditions.

FIG. 7 shows the effect of capsule configuration on caffeine release and probiotic survival.

FIG. 8 shows the effect of capsule configuration on cultivability of L. acidophilus strain after the stomach and small intestinal simulated digestion in fasted (A) and fed (B) conditions.

FIG. 9 shows the effect of probiotic administration through different capsules on microbial activity modulation in a simulated colonic environment.

### DEFINITIONS

As used herein, the terms “about,” “approximately,” or “generally,” when used to modify a value, indicates that the value can be raised or lowered by 10% and remain within the disclosed aspect.

The term “therapeutically effective amount” as used herein, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts capable of positively effecting the gut microbiome when the gut is the target for the active ingredient.

The term “nutraceutical” refers to any compound added to a dietary source (e.g., a food, beverage, or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion.

As used herein, the term “mammal” includes any mammal that may benefit from improved joint health, resilience, mood, recovery, and general health, and can include without limitation canine, equine, feline, bovine, ovine, or porcine mammals. For purposes of this application, “mammal” does include human subjects, and may be used interchangeably with animals.

As used herein, the term “capsule” means a conventional hard capsule, or a gelatin capsule intended for oral administration to a mammal. The capsule has two co-axial, telescopically joined parts, referred to as body and cap. Normally, caps and bodies have a side wall, an open end and a closed end. The length of the side wall of each of said parts is generally greater than the capsule diameter. The capsule caps and bodies are telescopically joined together so as to make their side walls partially overlap and obtain a capsule shell. “Partially overlap” also encompasses an embodiment wherein the side walls of caps and bodies have substantially the same length so that, when a cap and a body are telescopically joined, the side wall of said cap encases the entire side wall of said body. Thus, the capsules of the present invention do not structurally depart from the conventional definition of capsules. Generally, “capsule” refers to both empty and filled capsules whereas “shell” specifically refers to an empty capsule. In case the hard capsule shells are filled with substances in liquid form, it is intended that the hard capsules of the invention may be sealed or banded according to conventional techniques to avoid leakage of contained substances.

As used herein, the term “acid resistance” or “acid resistant” means that when subjected to the USP disintegration test, the capsule shells and capsules of the invention do not present leaks for at least 1 hour. For the purposes of this disclosure acid resistance is tested using the apparatus and procedure disclosed in the disintegration test for dosage forms of USP-30 (essentially, simulated gastric fluid TS, at 37±2° C. in a basket/rack assembly).

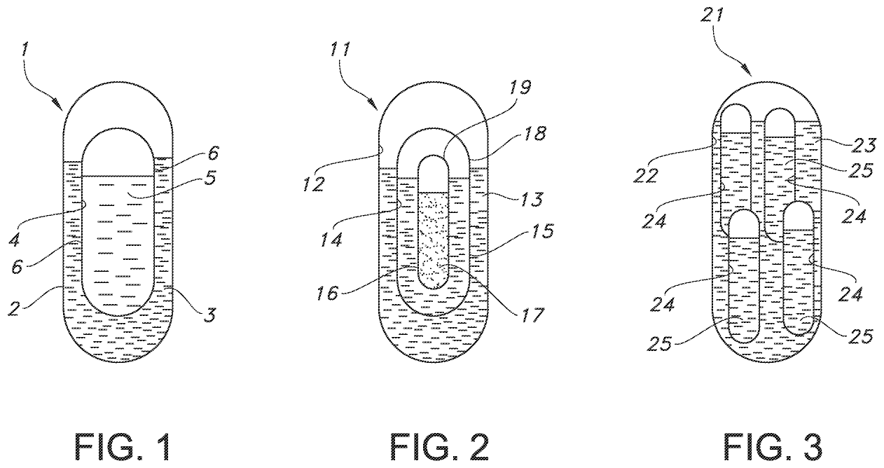

The acid resistant capsule shells and capsules of the invention also display satisfactory dissolution properties in simulated intestinal fluid at pH 6.8, 37±2° C., in a paddle apparatus. Dissolution profile of an exemplary capsule of the invention in simulated gastric and intestinal fluids is disclosed in the examples and FIG. 1. When tested in the dissolution test as disclosed in the Japanese Pharmacopoeia 2 (JP2), the hard capsules of the invention met the definition of enteric resistant hard capsules contained therein.

Other features and aspects of the present disclosure are discussed in greater detail below.

### DETAILED DESCRIPTION

It is to be understood by one of ordinary skill in the art that the present discussion is a description of exemplary embodiments only and is not intended as limiting the broader aspects of the present disclosure.

The capsules useable in the present disclosure may be hard capsules. Both the inner and outer capsules can be hard capsules.

Suitable hard capsules include capsules which may be prepared from a capsule forming aqueous composition containing a film forming polymer base material, optionally one or more colorants and water. Optionally, other additives may be present such as plasticizers, anti-bacterial-agents, gelling agents and neutralizing agents (particularly alkaline materials).

The film forming polymer base material may be selected from one or more celluloses, such as, for example, hydroxypropyl methylcellulose (HPMC or hypromellose), HPMCP, hydroxypropyl methyl cellulose acetate succinate (HPMCAS), methylcellulose (MC); gelatin; pullulan; polyvinyl acetate or poly vinyl alcohol and starch derivatives, for example, hydroxypropyl starch and mixtures thereof, as these film forming polymers form films with optimal mechanical performance in terms of elastic module and brittleness. In a particular embodiment, the film forming polymers comprise HPMC and/or gelatin. In another embodiment, the film forming polymers contains HPMC, which may be the sole film forming polymer base material. In a different embodiment, the film forming polymer base material may contain of gelatin as the sole film forming base material. Suitable types of HPMC are well-known in the art and an example is HPMC type 2910 (as defined in USP30-NF25). Other types of HPMC are HPMC 2208 and HPMC 2906 (as defined in USP30-NF25). A particular embodiment, the cellulose is hydroxypropyl methylcellulose (HPMC).

The HPMC methoxy and hydroxypropoxy contents herein are expressed according to the USP30-NF25. The viscosity of the HPMC 2% weight solution in water at 20° C. is measured according to the USP30-NF25 method for cellulose derivatives.

Typically, the aqueous composition comprises 10-50% by weight, based on the total weight of the capsule forming aqueous composition, of the film forming polymer and more typically between 15% and 35% by weight. Suitable hydroxypropyl methyl celluloses are commercially available. Generally, the film-forming polymer will represent the main constituent by weight of final capsule shells, after the capsule is formed and water is removed by drying. The use of water soluble polymers in dip molding manufacturing process for forming capsules is already known to the public and widely disclosed in many publications and patents. The capsule forming process is described in more detail below. The water soluble film forming polymers presently used are all commercially available.

In one particular embodiment, the HPMC in the aqueous composition herein is a HPMC having a viscosity of 4.0 to 5.0 cPs as a 2% w/w solution in water at 20° C. Viscosity of the HPMC solution in water can be measured by conventional techniques, e.g., as disclosed in the USP by using a viscometer of the Ubbelohde type. Suitable aqueous compositions can also be obtained by blending HPMCs of same type but different viscosity grade.

In an embodiment, the aqueous compositions used to make the capsules herein may contain between 0% and 5%, typically between 0% and 2% by weight based on the total weight of the capsule forming aqueous composition of additional non animal derived film-forming polymers typically used for the manufacture of hard capsules. In an embodiment, the HPMC aqueous compositions of the invention contain no other film-forming polymer beside the HPMC presently disclosed. Non-animal-derived film-forming polymers are for example polyvinyl alcohol, plant-derived or bacterial-derived film-forming polymers. Typical plant-derived film-forming polymers are starch, starch derivatives, cellulose, celluloses derivatives other than the HPMC as defined herein and mixtures thereof. Typical bacterial-derived film-forming polymer are exo-polysaccharides. Typical exo-polysaccharides are xanthan, acetan, gellan, welan, rhamsan, furcelleran, succinoglycan, scleroglycan, schizophyllan, tamarind gum, curdlan, pullulan, dextran and mixtures thereof.

In a one particular embodiment, the HPMC aqueous compositions herein contain between 0% and 1%, preferably 0% by weight based on the total weight of the capsule forming aqueous composition of animal-derived materials conventionally used for the manufacture of hard capsules. A typical animal-derived material is gelatin.

In another embodiment, the capsule forming aqueous composition herein up to about 10% by weight, based on the total weight of the capsule forming aqueous composition of a gelling agent or gelling system. Typically, the capsule forming aqueous compositions will contain between 0.2% and 5% by weight, based on the total weight of the capsule forming aqueous composition of a gelling agent or a gelling system. By “gelling systems” it is meant one or more cations with one or more gelling agents. Typical cations are K⁺, Na⁺, Lit, NH₄⁺, Ca⁺⁺, Mg⁺⁺ and mixtures thereof. Typical gelling agent(s) are hydrocolloids such as alginates, agar gum, guar gum, locust bean gum (carob), carrageenans, tara gum, gum arabic, ghatti gum, khaya grandifolia gum, tragacanth gum, karaya gum, pectin, arabian (araban), xanthan, gellan gum, konjac mannan, galactomannan, funoran, and mixtures thereof. As usually, gelling agents can be used in combination with cations and other ingredients such as sequestering agents to form a gelling system. Commercially available capsules usable in the present invention include, for example, capsules available from Lonza Consumer Health Inc, located in Greenwood, South Carolina, USA as VCaps®, VCaps® plus Color and PlantCaps®.

In a different embodiment, depending on the film forming polymer, the gelling agent or gelling system may be present in an amount of less than 0.2% by weight, based on the total weight of the capsule forming aqueous composition, and typically less than 0.1% by weight for a capsule forming composition which is essentially free of a gelling agent or system, and even 0% by weight, for a capsule forming aqueous composition which completely which is free of gelling system. When a gelling agent or gelling system is not used, the film forming polymer of the capsule forming aqueous composition must be capable of forming film without the need of a gelling agent.

In a particular embodiment, the HPMC aqueous compositions containing an on average HPMC Grade 2906 is suitable to give strong and physically stable gels without gelling systems, and the dissolution properties of the HPMC capsules made from are not adversely affected by the drawbacks typically associated with gelling systems, most notably cations. This type of capsule is available from Lonza Consumer Health Inc, located in Greenwood, South Carolina, USA as VCaps Plus®.

In another embodiment of the present disclosure, one of the capsules may be a delayed release capsule. Examples of delayed release capsules include those capsules which are acid resistant or are enteric capsules. These capsules do not dissolve in the stomach or under acidic conditions and allow the contents of the capsules to be delivered in the intestines of the user. Acid resistance may be achieved by coating a non-acid resistant capsule with an enteric film. The enteric film comprises well-known acid resistant materials that have a pH-dependent water solubility. Typically, these materials are carboxylic group-containing polymers, such as cellulose acetate phthalate (CAP), hydroxypropyl methylcellulose phthalate (HPMCP), hydroxypropyl methylcellulose acetate succinate (HPMC-AS), acrylic copolymers and shellac. These materials are water insoluble under gastric conditions (conventionally simulated by pH 1.2) and readily water soluble under intestinal conditions (conventionally simulated by a pH of 6.8). Drawbacks of the coating solution are typically represented by the complexity and costs of the manufacturing coating process, the high level of expertise needed to effectively perform it, the necessity to perform the coating at the end of the manufacturing cycle, i.e., once the capsules are already filled and, finally, the need for contacting the capsules with solvent-based coating compositions that may leave toxic solvent residues on capsule surface after drying.

So other methods of achieving acid resistance or enteric properties have been developed. In one aspect, the acid resistant capsules may be prepared from an aqueous composition for the manufacture of acid resistant hard pharmaceutical capsules, characterized in that it comprises (i) an aqueous solvent, (ii) gellan gum and (iii) one or more water soluble, film forming polymers, wherein the weight ratio of gellan gum to said one or more water soluble, film forming polymers is between 4/100 to 15/100 on a weight basis, lower and upper limits included.

In a particular embodiment, the capsule forming aqueous composition of the invention containing of (i) an aqueous solvent, (ii) gellan gum and (iii) one or more water soluble, film forming polymers, wherein the weight ratio of gellan gum to said one or more water soluble, film forming polymers is between 4/100 to 15/100, lower and upper limits included. The film forming polymers for the acid resistant or enteric capsules are the same as the film forming polymers described above. These acid resistant capsules are described in detail in U.S. Pat. No. 8,852,631 to Cade et al., which is hereby incorporated by reference.

The one or more water soluble film forming polymers contained in the capsule forming aqueous composition generally will represent the main constituent by weight of final capsule shells. The use of water soluble polymers in dip molding manufacturing process for preparing delayed release or enteric hard capsules is already known to the public and widely disclosed in many publications and patents. The water soluble film forming polymers presently used are all commercially available.

Gellan gum is an exopolysaccharide produced by fermentation. In the present invention, gellan gum is used in a ratio of about 4 to 15 parts, preferably about 4.5 to 8 parts, more preferably about 4.5 to 6 parts by weight, lower and upper limits included, per about 100 parts by weight of the one or more water soluble film forming polymers. In a different embodiment of the invention, gellan gum is used at a ratio of about 5 or 5.5 parts by weight per about 100 parts by weight of the one or more water soluble film forming polymers. Upon experimental evidence, It is believed that if lower amounts of gellan gum are used, the final hard capsules do not have enough acid resistance under disintegration test at pH 1.2, whereas higher gellan content, at process conditions (e.g. T and solid content) typical of conventional non-thermogelling dip-moulding techniques (for conventional processes, see e.g. the patent literature reported above), may cause excessive viscosity and excessive gelling ability of the aqueous composition thus making it impossible to manufacture the capsules at the requested high speed and quality. The preferred values of gellan to polymer ratio are believed to optimally combine the technical effects achieved by the present invention and processability aspects.

Due to its gelling properties, gellan gum is a typical component of setting systems conventionally used in the manufacture of immediate release hard capsules when the water soluble film forming polymers used, contrary to gelatin, do not present per se satisfactory gelling properties (e.g., HMPC or modified starches). However, in the prior art, gellan gum is used in amounts by weight which are typically very low with respect the weight of the water soluble film forming polymer(s). For example, amounts of gellan gum typically employed are below 1 part by weight per about 100 parts by weight of the water soluble film forming polymer(s), amount which are significantly lower than those used in the present invention,

Additionally, gellan gum is often used in combination with so-called gelling aids (typically salts of Na+, K+ or Ca2+). The use of low amounts of gellan gum and its combination with gelling aids are taught to fully respond to the need of making the main film forming polymer to gel on dipping pins and obtain suitable hard capsule shells.

It has been found that by working within the ratio of gellan gum to water soluble film-forming polymers as indicated above, suitable hard capsules can be obtained and also acid resistance can be imparted to such capsules.

Another remarkable advantage is that the addition of the so-called gelling aids is no longer necessary, even when working with film-forming polymers that, like HPMC, have per se poor gelling properties. In other words, when gellan gum is used in weight ratio indicated above, a composition suitable for the manufacture of hard capsules can be obtained out of HPMC or hydroxypropyl starch aqueous compositions without adding gelling aids (e.g., cations) to the aqueous composition. The optional absence of added gelling aids has an advantageous impact on stability of active ingredient filled into the final hard capsule shells and hard capsule dissolution profile. The fact that the aqueous composition of the invention does not contain added gelling aids preferably means that it does not contain gelling aids, e.g., cations, in an amount higher than the amount of the same aids that is naturally present in gellan gum. In another embodiment, the fact that the aqueous composition of the invention does not contain added gelling aids preferably means that it contains gelling aids, e.g., cations, in an amount not higher than the amount of the same aids that is naturally present in gellan gum. Such natural amount can be easily established by routine laboratory tests on purchased gellan gum batches or it can be directly provided by gellan gum suppliers.

The hard capsules of the with acid resistance do not leak at pH 1.2 in a USP-30 simulated gastric fluid for at least 1 hour, confirming the acid-resistant performance.

Typically, the combined amounts of ingredients (ii) and (iii) (i.e., gellan together with the one or more water-soluble film forming polymers) in the aqueous composition of the invention are between about 10% and 40%, more preferably between about 15% and 25% by weight over the total weight of the aqueous composition. Adapting the appropriate concentration of the film forming polymer to the specific polymer used and the desired mechanical properties of the film is well within the abilities of a skilled person in the field of hard capsules manufacturing. Commercially available delayed release capsules usable in the present invention include, for example, capsules available from Lonza Consumer Health Inc, located in Greenwood, South Carolina, USA as DRcaps®.

Optionally, the aqueous composition of the invention can contain at least one inert, non-toxic pharmaceutical grade or food grade pigment such as titanium dioxide, calcium carbonate iron oxides and other colouring agents. Generally, 0.001 to 5.0% by weight of pigment can be included in the aqueous composition. The weight is expressed over the total weight of the solids in the aqueous composition.

Optionally, the aqueous composition of the invention can contain an appropriate plasticizer such as glycerin or propylene glycol. To avoid an excessive softness, the plasticizer content has to be low, such as between 0% and 20%, more preferably between 0% and 10%, even more preferably between 0% and 5% by weight over the total weight of the solids in the aqueous composition.

Optionally, the aqueous composition of the invention can contain further ingredients typically used in the manufacture of hard capsules such as surfactants and flavoring agents in amounts known to a skilled person and available in publications and patents on hard capsules

In another aspect, the present invention relates to an acid resistant hard capsule shell obtained by using an aqueous composition as defined above. In a particular embodiment, the shell comprises (I) moisture, (II) gellan gum and (III) one or more water soluble film forming polymers, wherein the weight ratio of gellan gum to said one or more water soluble film forming polymers is between 4/100 to 15/100, lower and upper limits included.

In a preferred embodiment, the acid resistant hard capsule shell consists of (I) moisture, (II) gellan gum and (III) one or more water soluble film forming polymers, wherein the weight ratio of gellan gum to said one or more water soluble film forming polymers is between 4/100 to 15/100, lower and upper limits included. An exemplary commercially available capsule having acid resistance and a delayed release of the active ingredient is DRcaps® available from Lonza Consumer Health Inc, located in Greenwood, South Carolina, USA.

Whenever applicable and unless technically incompatible, all the features and preferred embodiments disclosed in connection with the aqueous compositions of the invention are disclosed also in connection with any other aspect of the invention, including the acid resistant hard capsule shells and shells of the invention.

The moisture content of the capsule shells of the invention mainly depends upon the one or more water-soluble film forming polymers used and relative humidity of the environment in which the shells are stocked after production. Typically, the moisture content is between about 2% and 16%, over the total weight of the shell. As an example, under conditions conventionally adopted for storing hard capsules, the hard capsule shells of the present invention contain between about 2-8%, preferably about 2-6%, preferably about 3-6% by weight of moisture over the weight of the shell when the only film forming polymer used is HPMC and 10-16% of moisture over the weight of the shell, when the only film forming polymer used is gelatin.

In another aspect, the present invention relates to an acid resistant hard capsule comprising a shell as defined above.

The capsules of the invention can be obtained by filling the shells of the invention with one or more substances to be encapsulated. Once filled, the capsules can be made tamper-proof e.g., by using appropriate banding solution used in the field of hard capsules to make the joint permanent.

In a particular embodiment, a hard capsule shell of the invention as defined above is filled with one or more acid-instable substances and/or one or more substances associated with gastric side effects in humans and/or animals.

In another aspect, the present invention relates to a dip-molding process for the manufacture of an acid resistant hard pharmaceutical capsule shell, said process comprising the steps of:
(a) dipping pins into an aqueous composition as defined above
(b) withdrawing the dipping pins from the aqueous composition and
(c) drying the composition adhered on the dipping pins so as to obtain a shell;
wherein the steps (a) to (c) are performed in the order in which they are presented.

After drying step (c), the shell obtained can be stripped off the pins and cut to a desired length. In this manner capsule shell parties (bodies and caps) are obtained that subsequently can be telescopically joint so as to form a final empty capsule. In case of filling with liquid substances, and if desired, once filled the capsule can be made tamper-proof by appropriate techniques known in the field such as banding or sealing techniques, including those described in U.S. Pat. Nos. 9,579,290; and 9,980,918; and U.S. Patent Application 2020/0163893, each hereby incorporated by reference in their entirety.

Referring firstly to FIG. 1, a double capsule delivery device, indicated generally at 1, comprises a first, outer hard capsule 2 containing a liquid active principle 3 and a second, inner hard capsule 4 which also contains the same liquid active principle 5 as that contained in the outer hard capsule 2 and may be coated, as shown at 6.

Similarly, FIG. 2 illustrates a triple capsule delivery device, indicated generally at 11, which comprises a first, outer hard capsule 12 containing a liquid active principle 13 and a second, inner hard capsule 14 which also contains the same liquid active principle 15 as that contained in the outer hard capsule 12 and may be coated, as shown at 18. The second, inner hard capsule 14 also contains a third, inner hard capsule 16 which, in turn, contains the same active principle as that contained in the first and second, outer and inner hard capsule 12 and 14 but in solid particulate form. That second, inner hard capsule 14 may also be coated, as shown at 19. The capsules 12, 14 and 16 are in series with each other.

Referring now to FIG. 3, a multiple capsule delivery device, indicated generally at 21, comprises a first, outer hard capsule 22 containing a liquid active principle 23 and four inner hard capsules 24 containing an active principle 25 which is the same as that contained in the first, outer capsule 22 but in semi-solid form. The four inner capsules 24 are in parallel with each other but in series with the outer capsule 22.

Exemplary Active Substance that can be effectively delivered to the mammal, includes nutraceuticals, pharmaceuticals, probiotics and combinations thereof. The present disclosure is very effective in allow probiotics survive past the stomach when ingested.

It has been shown that a capsule-in-capsule configurations can be used to delivery active substances to a mammal in need of treatment to the lower intestines. It has been discovered that using an acid resistant inner capsule, inside an outer capsule, whether the outer capsule is HPMC with a gelling agent, thermogelled HPMC or gelatin or an acid resistant capsule, as compared to the acid resistant capsule alone. In a thermogelled outer capsule and an acid resistant inner capsule, it has been shown that the amount of the active ingredient delivered to the intestines, and in particular the lower intestines, can increase by 10 times, 20 times or more. In the case of an acid resistant inner and outer capsules, the amount of the release of the active ingredient can be 20 to 50 times or more greater than a single acid resistant capsule, whether the mammal is in a fasted or fed state.

The advantages of the present disclosure and the delivery system used to deliver active substances to a mammal and provide benefits, such as allowed significant increase in lactate production, indicative of high fermentation and consequently functional probiotic, a significant decrease in propionate under fed conditions, suggesting decrease in propionate-producing bacteria, increase in butyrate production under fasting conditions, indicative of increase in butyrate-producing bacteria and provide a functional changes suggest a change in the microbiome diversity in the intestines. The combination of capsules suggested herein can be used to target release active substances to the intestines of a user and may provide increase bioavailability of active substances to the mammal.

Nonetheless, certain embodiments of the present disclosure may be better understood according to the following examples, which are intended to be non-limiting and exemplary in nature.

### EXAMPLES

### Example 1

### Test Procedure

All the reagents used in these examples were provided by Sigma (Overijse, Belgium), otherwise stated.

### Composition of Capsule Systems

Seven types of capsule-in-capsule systems and three single capsules were evaluated in this Example (Table 1). The configuration of the capsule-in-capsule was a combination of outer capsules (size #00) and inner capsules (size #3) as follows:

Capsules were filled with caffeine (50 mg/capsule) as a marker for release and a probiotic strain (L. acidophilus ATCC-43121, LGC Standards) at a concentration of 2×10¹⁰ CFU/capsule as indicated in Table 1.

TABLE 1
Capsule configurations

| | | | Outer | | | |
|---|---|---|---|---|---|---|
| | | Outer Capsule | Capsule | | | |
| Sample | Configuration | (all size #00 | Fill | Inner Capsule | Caffeine | Probiotic Strain |
| A | Capsule-in- | acid resistant | Glycerol | thermogelled | yes | L. acidophilus |
| | capsule | capsule HPMC | | HPMC capsule | | LA-14 |
| | | capsule | | | | |
| B | Capsule-in- | acid resistant | Glycerol | HPMC capsule | yes | L. acidophilus |
| | capsule | capsule HPMC | | with gelling | | LA-14 |
| | | capsule | | agent | | |
| C | Capsule-in- | acid resistant | Glycerol | acid resistant | yes | L. acidophilus |
| | capsule | capsule HPMC | | capsule HPMC | | LA-14 |
| | | capsule | | capsule | | |
| D | Capsule-in- | HPMC capsule | Glycerol | HPMC capsule | yes | L. acidophilus |
| | capsule | with gelling | | with gelling | | LA-14 |
| | | agent | | agent | | |
| E | Capsule-in- | HPMC capsule | Glycerol | thermogelled | Yes | L. acidophilus |
| | capsule | with gelling | | HPMC capsule | | LA-14 |
| | | agent | | | | |
| F | Capsule-in- | HPMC capsule | Glycerol | acid resistant | Yes | L. acidophilus |
| | capsule | with gelling | | capsule HPMC | | LA-14 |
| | | agent ® | | capsule | | |
| G | Capsule-in- | Gelatin capsule | Fish Oil | Banded acid | Yes | L. acidophilus |
| | capsule | | 18/12 + | resistant | | LA-14 |
| | | | Silica | capsule HPMC | | |
| | | | | capsule | | |
| Comparative | Single capsule | acid resistant | None | None | Yes | L. acidophilus |
| C1 | | capsule HPMC | | | | LA-14 |
| | | capsule | | | | |
| Comparative | Single capsule | HPMC capsule | None | None | yes | L. acidophilus |
| C2 | | with gelling | | | | LA-14 |
| | | agent | | | | |
| Comparative | Single capsule | thermogelled | None | None | Yes | L. acidophilus |
| C3 | | HPMC capsule | | | | LA-14 |

### Testing of Capsule Configurations

Testing of the capsule configuration was completed in two phases. The first phase was upper GIT simulation. And a second phase of testing was completed on complete.

The upper GIT simulation was performed in two sequential double-jacketed reactors simulating the stomach and small intestine digestion conditions. The temperature was maintained at 37° C. and continuous magnetic stirring (300 rpm) was applied during the experiments. Capsules were maintained in the stomach and small intestinal reactors with specially designed sinkers for capsule dissolution studies.

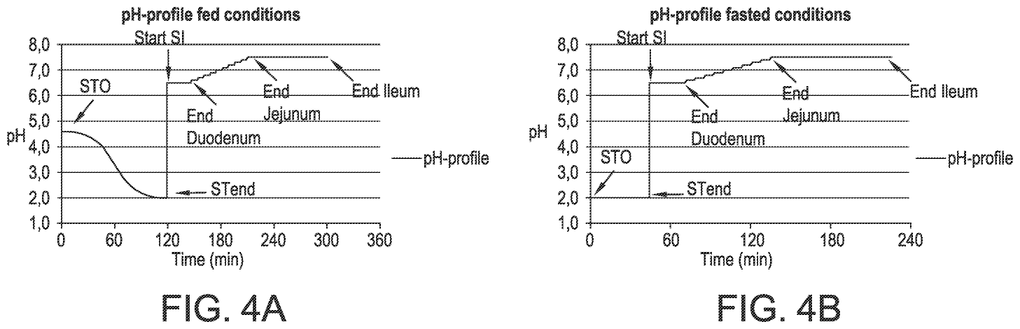

To mimic fed (i.e., consumption of the product during or immediately after a meal) and fasted (i.e., consumption of the product before a meal) conditions, the pH profile, enzyme levels and retention times were adjusted. Shown in FIG. 4 is the pH-profile during the experiments under fed (A) and fasted (B) conditions. The pH of the medium was controlled automatically. Arrows indicate the start and end of the stomach (STO, STend) and small intestinal incubation phase. The differences are shown in FIGS. 4A and 4B, where FIG. 4A shows the pH profile for fed conditions and FIG. 4B shows the pH profile for fasted conditions. As shown, the pH-profile during the experiments under fed (A) and fasted (B) conditions is different. ST=stomach; SI=small intestine. The pH of the medium was controlled automatically. Arrows indicate the start (STO) and end of the stomach (STend) and small intestinal incubation phase (Start SI, End duodenum, end Jejunum and end Ileum).

Under fasted conditions, the stomach digestion was simulated with a 45 min incubation in a gastric fluid (76 mL, pH 2) containing KCl 0.66 g/L, NaCl 3.63 g/L and mucin 3.95 g/L, 0.4 mL of lecithin (Carl Roth GmbH+Co. KG, Germany) (3.4 g/L) and 3.6 mL pepsin (Chem Lab, Zedelgem, Belgium) (10 g/L). Continuous pH control was performed by a Senseline PH meter F410 (ProSense, Oosterhout, The Netherlands) and an automatic pump dosage of HCl (0.5M) or NaOH (0.5M) to keep the pH constant at 2. After the stomach incubation, the gastric digestion volume was measured and adjusted to 100 mL with MilliQ water. Capsule sinkers and gastric fluids were transferred to the small intestine reactors and 35.2 mL pancreatic juice (NaHCO₃2.6 g/L, Oxgall 4.8 g/L and pancreatin 1.9 g/L), 2.15 mL trypsin (10 g/L) and 2.7 mL chymotrypsin (10 g/L) were added. The small intestine pH was gradually increased from 2 to 6.5 and maintained at this pH over a 27 min period, simulating the duodenal incubation. This phase was followed by a stepwise pH increase (0.1 pH units every 7 min) to 7.5 within a 63 min period, mimicking the jejunal environment. Finally, the pH remained constant at 7.5 during a 90 min ileal incubation. The pH increase was achieved by the addition of NaHCO₃ (8.4 g/L) at 60, 90 and 120 min, mimicking the dilution of the intestinal contents.

Under fed conditions, testing was carried out in similar way than the fasted conditions with the following modifications. The stomach digestion was simulated with a 120 min incubation in a solution of 76 mL of gastric juice containing the SHIME® nutritional medium (PDNM001B 20.53 g/L, ProDigest, Ghent, Belgium), NaCl (3.63 g/L), KCl (0.65 g/L), 0.4 mL lecithin (13.5 g/L) and 3.6 mL pepsin (40 g/L) at pH 4.6. During the fed-stomach digestion, a sigmoidal decrease of the pH from 4.6 to 2 was obtained by a controlled pump of HCl (0.5M) at established time points. After the stomach incubation, a small intestinal phase was performed as described before, but with different compositions of pancreatic juice (NaHCO₃7.7 g/L, oxgall 15 g/L and pancreatin 10 g/L), 2.15 mL trypsin (10 g/L), 2.7 mL chymotrypsin (10 g/L). The pH increase was achieved by adding NaHCO₃ (4.8 g/L) at 60, 90 and 120 min.

A blank control capsules without caffeine or L. acidophilus was included in all the assays as a background media for the caffeine HPLC analysis. The negative control consisted of L. acidophilus and caffeine used alone without capsules. All the assays were performed in triplicate.

### Whole Gastrointestinal Tract Simulation and Colonic Fermentation

In a second phase of testing and entire GIT test was completed. Following the upper GIT incubations under fed and fasted conditions, as described above, a colonic incubation was simulated by addition of 160 mL fresh colonic anaerobic medium [KH2PO4 (6.6 g/L), K2HPO4 (20.5 g/L), NaCl (5 g/L), yeast extract (2 g/L), peptone (2 g/L), glucose (1 g/L), starch (2 g/L), mucin (1 g/L), L-cysteine HCl (0.5 g/L), Tween® 80 (2 mL)], 40 mL of anaerobic PBS [K2HPO4 (8.8 g/L), KH2PO4 (6.4 g/L), NaCl (8.5 g/L) and L-cysteine HCl (0.5 g/L)]. A fixed pH interval between 6.5 and 5.8 was implemented and automatically adjusted by adding HCl (0.5M) or NaOH (0.5M). Next, a fecal inoculum derived from a healthy donor was used to inoculate the colonic incubation, as previously described.

Briefly, a mixture of 1:10 (w/v) of fecal sample and anaerobic phosphate buffer (K2HPO4 8.8 g/L; KH2PO4 6.8 g/L; sodium thioglycolate 0.1 g/L; sodium dithionite 0.015 g/L) was homogenized for 10 min (BagMixer 400, Interscience, Louvain-La-Neuve, Belgium). After centrifugation (2 min, 500 g) (Centrifuge 5417C, Eppendorf, VWR, Belgium), large particles were removed and the fecal inocula was added to the different reactors at 20% (v/v) to the upper GIT digestion fluids. Colonic incubations were performed under anaerobic conditions at 37° C., and 90 rpm agitation during 24 h (MaxQ 4000 Benchtop Orbital Shaker, Thermo Fisher Scientific, Belgium).

### Caffeine Release Quantification

Caffeine was quantified by HPLC-UV/Vis (Hitachi Chromaster HPLC-DAD, Hitachi High-Tech Corporation, Japan) using an isocratic separation method (25% methanol:75% water) on a Kinetex® C18 LC column (serial number 00D-4601-E0; 5 μm, 100 Å, LC Column 100×4.6 mm, solid support of Core-shell Silica) (Phenomenex, Belgium). The column temperature was kept controlled at 25+0.1° C. The total run time per sample was 7 min. The injection volume was 10 μL and the UV/Vis detector was operated at 272 nm. Quantification of caffeine was performed using external standards (Sigma-Aldrich, Merck KGaA, Darmstadt, Germany). Prior to injection on the column, the samples were centrifuged for 15 min at 5000 g. Subsequently, the supernatant was filtered through a 0.2 μm filter into HPLC vials. Caffeine analysis was performed on gastric samples at 15, 30 and 45 min (fed and fasted) and 60, 90 and 120 min (fed). Small intestinal samples were collected at 30, 60, 90, 120, 150 and 180 min. Colonic samples were obtained at 1, 2 and 24 h of incubation.

L. acidophilus Survival by PMA-Based qPCR

Bacterial survival was tested by propidium monoazide (PMA) based qPCR. For this procedure 1:1 (v/v) dilution of sample in anaerobic phosphate buffer was mixed with 1.25 μL PMAxx™ dye (20 mM) (VWR International Europe, Leuven, Belgium). Samples were incubated 5 min in constant shaking (500 rpm) in the dark and centrifuged at max. speed (18.327 g) for 30 sec. Subsequently, the samples were placed in the PhAST blue PhotActivation System (GenIUL, Barcelona, Spain), a LED-active Blue system (GenIUL, Barcelona, Spain), for 15 min and centrifuged 10 min at 13.000 g. The supernatant was immediately removed, and DNA was isolated as described before. The qPCRs were performed with specific primers for Lactobacillus acidophilus [L.acid_F (5′-GAAAGAGCCCAAACCAAGTGATT-3′) and L. acid_R (5′-CTTCCCAGATAATTCAACTATCGC-3′)] using a QuantStudio 5 Real-Time PCR system (Applied Biosystems, Foster City, CA, USA) with the program conditions previously described in (Van den Abbeele, Kamil et al. 2018). L. acidophilus survival was tested at the end of the stomach incubations (45 min for fasted condition and 120 min for fed condition), at 60, 120 and 180 min of the small intestinal digestion and at 1, 2 and 24 h of colonic fermentation.

L. acidophilus Cultivability

In samples obtained during the entire gastrointestinal tract passage, L. acidophilus cultivability was tested through MRS agar plating. Samples were collected at the end of the gastric (45 min for fasted and 120 min for fed) and small intestinal phase (180 min) and a ten-fold dilution series in anaerobic phosphate-buffered saline were plated in MRS agar plates. Plates were incubated aerobically at 37° C. for at least 48 h. The number of colony-forming units (CFU) is reported as average log (CFU)±SEM (n=3).

Evaluation of the L. acidophilus function and the metabolic activity of gut microbiota under colonic conditions

During 24 h of colonic incubation, samples at time point 0, 1, 2 and 24 h were obtained for microbial activity assessment. The pH measurements were performed using a Senseline pH meter F410 (ProSense, Oosterhout, The Netherlands). Short chain fatty acids (SCFA) (acetate, propionate, and butyrate) and branched chain fatty acids (BCFA) (isobutyrate, isovalerate, and isocaproate) were determined by gas chromatography as previously described (Ghyselinck, Verstrepen et al. 2020). Lactate production was assessed with a kit (R-Biopharm, Darmstadt, Germany), according to the manufacturer's instructions.

### Statistical Methods

Results are presented of the mean and standard error of the mean (SEM) from triplicates. Two-way ANOVA tests including time and different conditions were applied, with t-Tukey test for multiple comparisons. Statistical differences were set as a p<0.05. Analysis was performed using GraphPad Prism software, version 9.0 (GraphPad Software, CA, USA).

### Results

Characterization of capsule release behavior during the upper gastrointestinal tract passage under fed or fasted conditions

In the first part of the study, the 10 capsule configurations shown in Table 1 were subjected to passage in upper GIT simulation under fasted and fed conditions. Caffeine was used as an active marker to evaluate the capsules dissolution at different time points during gastric and small intestinal digestion-like environment.

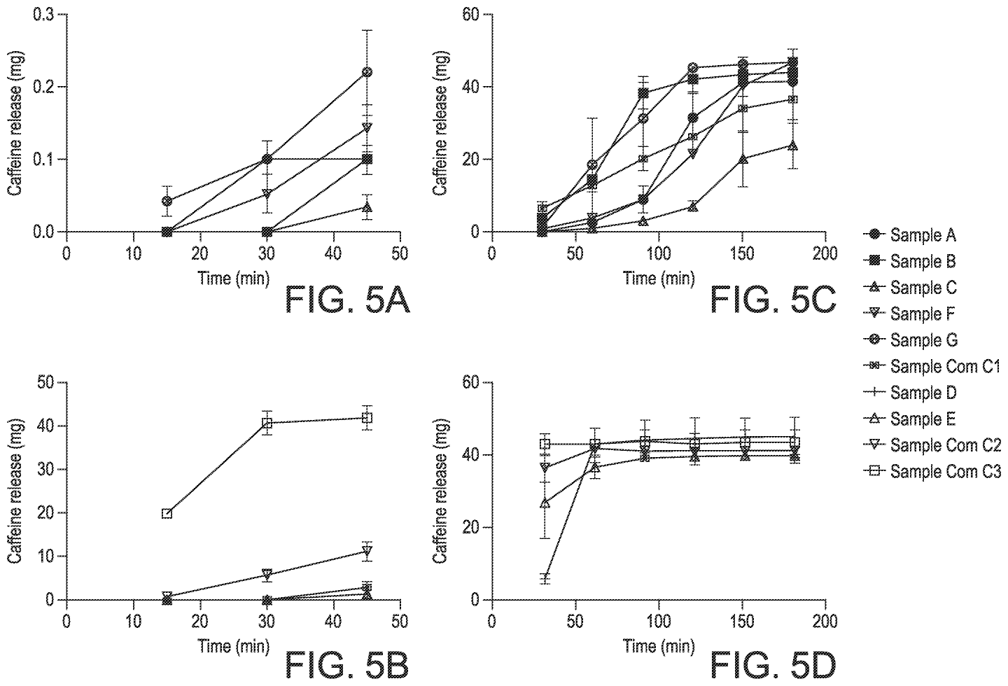

FIG. 5 shows the effect of capsule configuration on caffeine released during the fasted conditions as described above. The left panel of FIG. 5, left panel shows caffeine release in the stomach. After 15 min of gastric digestion, there was a release of caffeine from the Comparative Sample C3 (19.7±1.3 mg) and in to lesser extend for Comparative Sample C2 (0.7±0.3 mg) and Comparative Sample C1 (0.2±0.04 mg). After 30 minutes of incubation, the free caffeine for Comparative Sample C3 increased rapidly (40.8±2.6 mg), and in to lesser extend for Comparative Sample C2 (5.7±1.4 mg), Comparative Sample C1 (0.7±0.1 mg). Sample E (0.5±0.1 mg), Sample A (0.1±0.003 mg) and Sample G (0.1±0.02 mg). At the end of the stomach incubation (45 min), Comparative Sample C3 had the highest caffeine release (41.9±2.8 mg), showing a complete dissolution of the capsule. Other capsules displayed a partial caffeine release, with values of 11.3±2.2 mg for Comparative Sample 2, 2.9±1.5 mg for Sample E and 1.5±0.3 mg for Comparative Sample 1. Finally, low values of caffeine release (0.1-0.2 mg), indicating a high integrity of the capsule, were found for Sample G, Sample F, Sample A, Sample D, Sample C and Sample B.

The right panel of FIG. 5 show caffeine release in the intestine under simulated digestion conditions described above. At the end of the duodenal incubation, there was a strong increase in caffeine release for Comp Sample C2 (36.4±3.9 mg) and Sample E (27.0±9.8 mg) and a small but steady release for Comp Sample C1 (6.5±1.5 mg), Sample D (5.8±1.4 mg), Sample B (3.8±0.6 mg), Sample G (1.7±0.5 mg), Sample F (1.0±0.1 mg) and Sample A (0.5±0.2 mg). Sample C capsules remained intact (0.2±0.1 mg), while the caffeine from the Comp Sample C3 was already released during the stomach incubation. After 60 minutes of the small intestine incubation, there was a strong increase in caffeine release for Sample D (42.6±4.8 mg) and Sample E (36.5±3.0 mg), indicating a complete dissolution of the capsules in the middle of jejunal phase. Comp Sample C2 were also completely disintegrated (41.8±2.3 mg). Slow but steady release continued for Sample G (18.5±13 mg), Sample B (14.3±1.2 mg), Comp Sample C1 (12.7±1.7 mg) and Sample F (3.8±0.1 mg) and Sample A (2.5±0.9 mg). The first release of caffeine was detected for Sample C (0.9±0.2 mg) in the jejunal incubation. At the start of the ileal phase, after 90 minutes of small intestinal incubation, there was a strong increase in caffeine release for Sample B (38.2±4.6 mg) and Sample G (31.1±9.8 mg), and slower values for Comp Sample C1 (20.1±3.4 mg), Sample A (8.8±3.8 mg), Sample F (8.8±0.1 mg), Sample C (30.1±0.7 mg). Further in the ileal phase, after 120 minutes of small intestinal incubation, Sample B and Sample G were completely dissolved. A strong increase in caffeine release was observed for the Sample A capsule (31.6±6.6 mg). The other capsules, i.e., Comp Sample C1 (26.2±4.7 mg), Sample F (21.4±0.6 mg) and Sample C (7±1.7 mg), still showed high integrity, with a low and continuous caffeine release, until the end of the small intestinal phase. Partially dissolved capsules at the end of the incubation were Sample C (24.2±6.8 mg), while Sample F (46.3±4.2 mg), Sample A (41.5±0.4 mg) and Comp Sample C1 (36.7±6.5 mg) were completely dissolved.

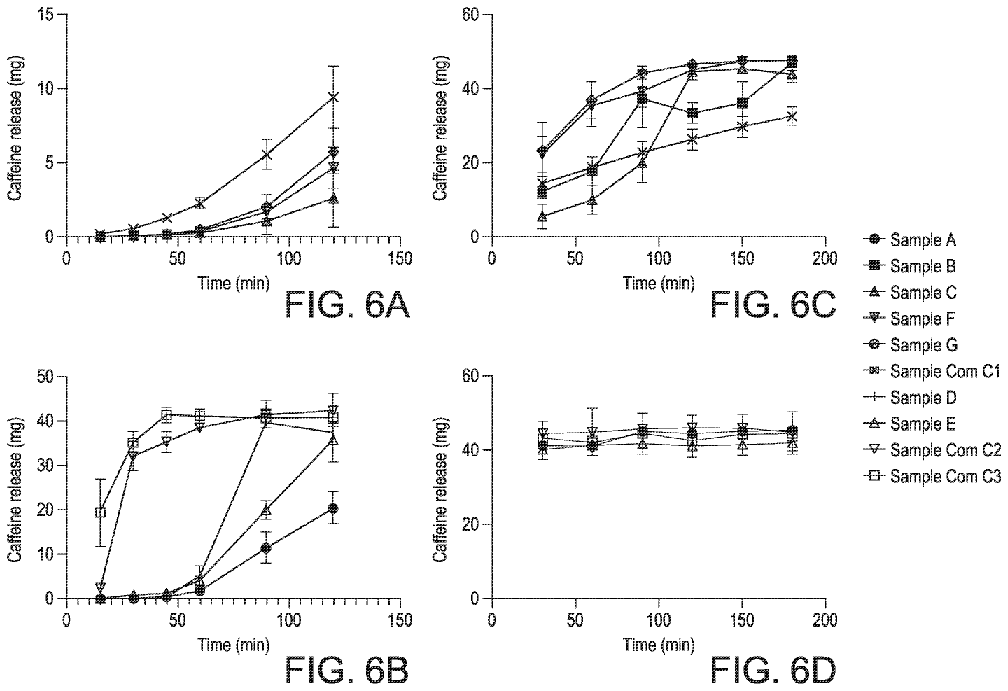

FIG. 6 shows the effect of capsule configuration on caffeine released during the fed conditions, as described above. The left panel of FIG. 6 show the results of caffeine release in the stomach under fed conditions. During the fed incubation (FIG. 6, left panel), and after 15 min of stomach digestion, caffeine was detected in Comp Sample C3 (19.5±7.6 mg) and Comp Sample C2 (2.4±1.4 mg), while after 30 minutes of gastric digestion, only a small caffeine release (0.1-0.9 mg) occurred for Sample E, Comp Sample C1, Sample A, Sample D, Sample F and Sample G. A high increase of caffeine was observed for Comp Sample C2 (32.2±3.3 mg) and Comp Sample C3 (35.3±2.5 mg). After 45 minutes, the Comp Sample C3 capsule was dissolved. The Comp Sample C2 capsule released 35.4±2.3 mg. Other capsules showed a slow but steady release; Sample E (1.3±0.7 mg), Comp Sample C1 (1.3±0.1 mg), Sample A (0.5±0.1 mg), Sample D (0.4±0.1 mg), Sample F (0.2±0.01 mg) and Sample G (0.2±0.1 mg), with Sample B and Sample C showing a first sign of caffeine release (0.1±0.003 mg).

Mid stomach incubation (60 min), the Comp Sample C2 capsule is completely dissolved. Slow but steady release continued for the following capsules: Sample D (5.2±2.3 mg), Sample E (4.3±1.0 mg), Sample A (1.9±0.1 mg), Comp Sample C1 (2.3±0.4 mg), Sample B (0.5±0.1 mg), Sample G (0.5±0.2 mg), Sample F (0.4±0.1 mg) and Sample C (0.3±0.2 mg). After 90 minutes of the stomach incubation, a strong increase in caffeine release occurred for Sample D (39.8±0.1 mg), indicating a complete dissolution of the capsule, and in a lesser extend for Sample E (20.1±2.1 mg) and Sample A (11.7±3.5 mg). Slow but steady release continued for Comp Sample C1 (5.6±1.1 mg), Sample B (2.8±0.6 mg), Sample G (2.1±0.8 mg), Sample F (1.7±0.5 mg) and Sample C (0.2±0.01 mg). At the end of the stomach incubation, the Sample E capsule was completely dissolved. The other capsules were partially dissolved: Sample A (20.6±3.6 mg), Comp Sample C1 (9.5±2.1 mg), Sample B (6.2±1.0 mg), Sample G (5.8±1.5 mg), Sample F (4.7±1.4 mg) and Sample C (0.7±0.04 mg).

The right panel of FIG. 6 show the results of caffeine release in the small intestine under fed conditions. The small intestinal incubation started with four completely dissolved capsules: Comp Sample C3, Sample D, Sample E and Sample B. After the duodenal incubation, the Sample A capsule was also completed dissolved (41.4±0.7 mg). The slow caffeine release continued in the small intestine for Sample G (23.7±7.3 mg), Sample F (22.4±4.8 mg), Comp Sample C1 (14.5±3.1 mg), Sample B (12.3±1.9 mg) and Sample C (5.5±0.1 mg). In the jejunal phase (60 min of small intestinal digestion), caffeine release of Sample G (37.1±5.0 mg) and Sample F (35.7±5.9 mg) was complete, while Comp Sample C1 (18.8±3.1 mg), Sample B (17.7±3.9 mg) and Sample C (6.3±3.8 mg) showed higher integrity and lower caffeine release to the digestion media. In the ileal phase (90 min of small intestine digestion), Sample G and Sample F were completely dissolved. There was a strong increase in caffeine release of the Sample B (37.4±7.8 mg) at the start of the ileal phase, indicating a complete dissolution of the capsule. Comp Sample 1 (22.8±3.1 mg) and Sample C (15.8±3.2 mg) continued a slow caffeine release until 120 minutes of small intestine incubation, when all the caffeine contained in Sample C was present in the digestion fluids (44.5±2.0 mg), indicating a complete disintegration of the capsule. Only the Comp Sample C1 continued their slow and steady caffeine release throughout the further incubation until a final release of 32.7±2.5 mg of caffeine at the end of the small intestinal incubation. Based on the findings of the first phase of the study, a second phase of the study was completed with Sample B, Sample C and Comparative Sam

Protection of L. acidophilus by Sample C and Sample B During the Stomach and Small Intestinal-Like Environment Digestion Promote Probiotic Survival at Colonic Level.

In the second phase of the study, three capsule configurations (Sample C, Sample B and Comp Sample C3) were selected based on their delayed release in the first part of the testing, to evaluate their behavior during the full gastro-intestinal tract under fasted or fed conditions. L. acidophilus survival and its modulatory effect in a colonic ecosystem were further tested. Comp Sample C3 was selected as this was the most immediate release capsule and could be used as a control. Sample C was selected as this was the capsule-in-capsule configuration with the most delayed release of caffeine in the upper GI sections in fasted and fed conditions. The third capsule, Sample B, was selected as the second most delayed capsule-in-capsule configuration in fed conditions.

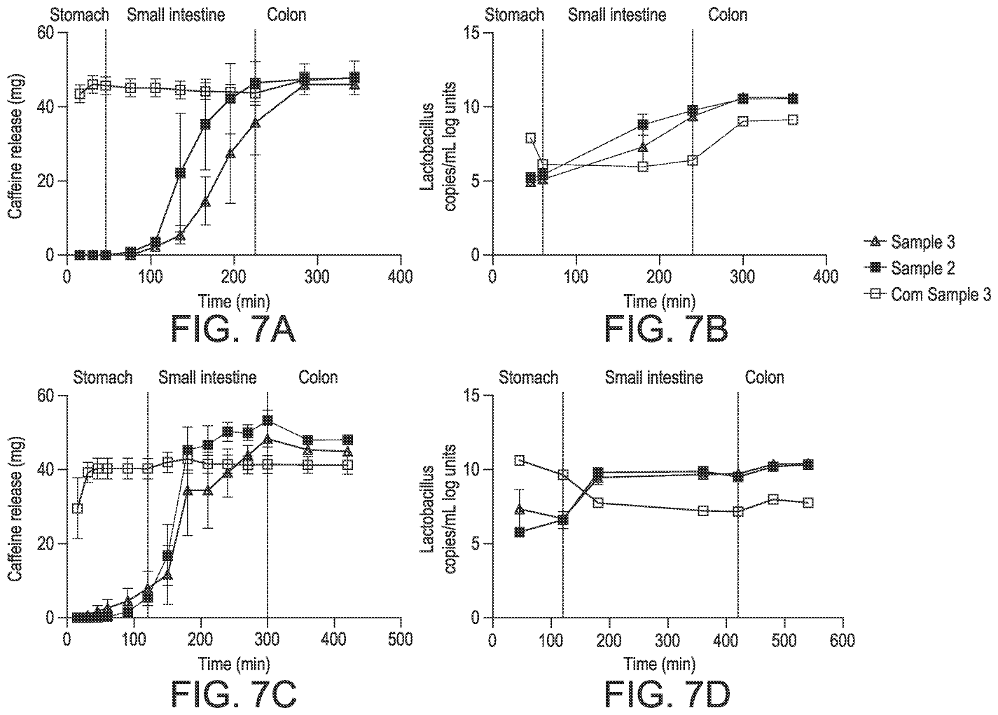

Effect of capsule configuration on caffeine release and probiotic survival are shown in FIG. 7. FIG. 7A and FIG. 7C show the time-course of caffeine release during the gastric, small intestine and colonic digestion. FIG. 7A is in a fasted condition and FIG. 7C is in a fed condition. Dots represent caffeine content in the corresponding digestion or fermentation media at selected time points (mean±SEM, n=3). FIG. 7B and FIG. 7D show the time-course of Lactobacillus acidophilus survival in the gastric, small intestine and colonic digestion. FIG. 7B is in a fasted condition and FIG. 7D is in a fed condition. Dots represent copies/mL in log units of PMA treated samples in the corresponding digestion or fermentation media at selected time points (mean±SEM, n=3).

As previously observed, in fasted conditions, caffeine release was significantly faster for Comp Sample C3 than for dual configurations (FIG. 7A), indicating a disintegration of the capsule before arriving at the colonic environment. At the end of the small intestine incubation time, Sample C were partially dissolved, with a complete capsule dissolution after one hour of colonic incubation.

At the end of the stomach incubation, PMA-DNA copies L. acidophilus (FIG. 7B) were similar for both Sample B (log 5.2±0.1 copies/mL) and Sample C (log 5.0±0.2 copies/mL), while higher PMA-DNA copies were detected for Comp Sample C3 (log 7.94 copies/mL), likely due to higher release of the probiotic strain into the digestion fluid. However, after 60 minutes of small intestinal incubation, this number was reduced to log 6.2±0.3 copies/mL, while for the other capsules, PMA-DNA copies remained within similar values. After 120 minutes of small intestinal incubation, the PMA-DNA copies L. acidophilus were log 8.8±0.7 copies/mL for Sample B and log 7.4±1.2 copies/mL for Sample C, indicating a high survival of the strain until the end of the small intestinal conditions.

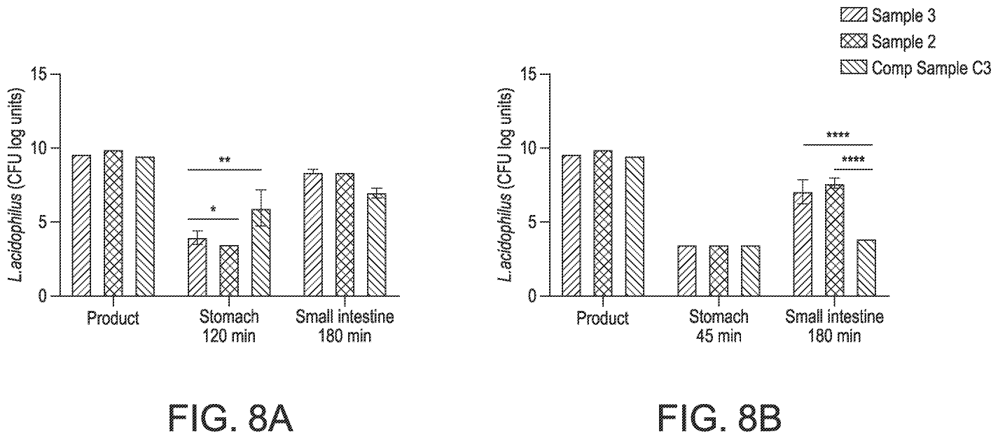

L. acidophilus survival based on its growth on agar plates after gastric and intestinal passage is presented in FIG. 8. FIG. 8 shows the effect of capsule configuration on cultivability of L. acidophilus strain after the stomach and small intestinal simulated digestion in fasted (A) and fed (B) conditions. Bars represent CFU in log units (mean±SEM, n=3) obtained by plate counting from the stomach and small intestinal digestion fluids exposed to different capsule configurations. Product refers to the maximum L. acidophilus CFUs inoculated in the different capsules. Significant differences are marked with asterisks (p<0.05 *, p<0.01 **, p<0.001 ***, p<0.0001 ****) L. acidophilus from Sample C and Sample B showed a significantly higher growth than when it is included in Comp Sample C3 under fasted conditions in the small intestinal environment, while in the fed state, the difference was observed in the stomach phase after 120 min, with slower colony forming units (CFU) in Sample C and Sample B than in Comp Sample C3. This is likely caused by a higher release of capsule contents from Comp Sample C3 on stomach medium.

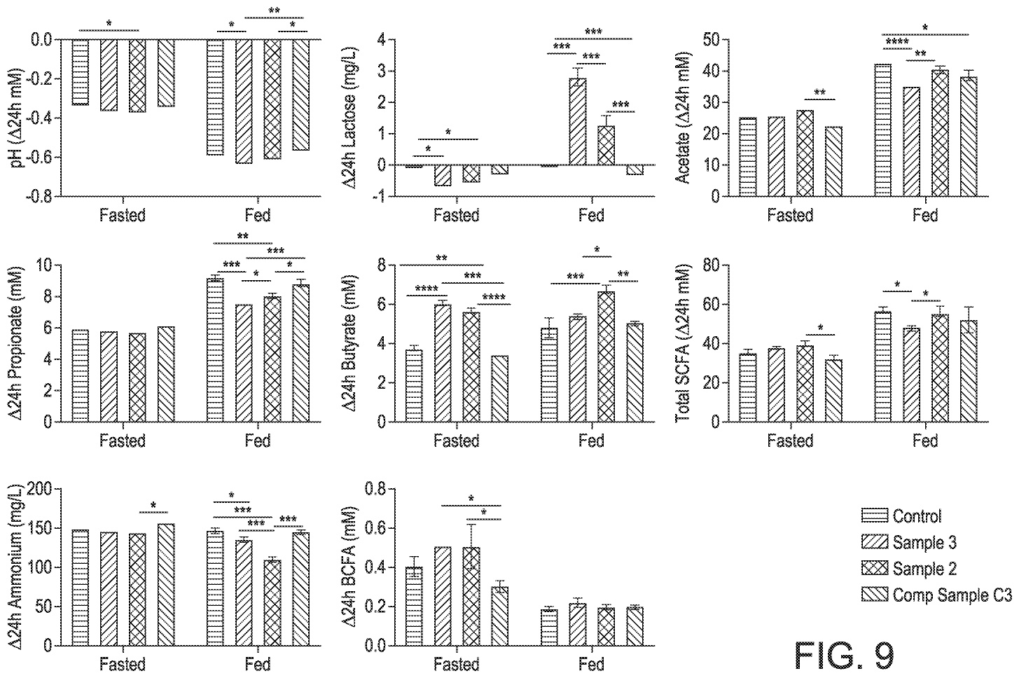

In the simulated colon environment, the probiotic administration's effect on the microbial activity via the three capsules was measured (FIG. 9) at different time points. In general, less effect was observed under fasted than fed conditions. Butyrate was the most affected metabolite.

Under fasted conditions, the only significant difference was observed between Sample B and Comp Sample C3 (FIG. 9). Specifically, butyrate was increased when L. acidophilus supplementation was included in Sample C (6.0±0.3 mM) and Sample B (5.6±0.3 mM) compared to the control Comp Sample C3 (3.4±0.1 mM). Ammonium levels slightly increased with Comp Sample C3 (156.1±6.1 mg/L) compared to Sample B (143.8±1.7 mg/L) while BCFA showed the opposite trend, with a significant decrease in Comp Sample C3 (0.3±0.01 mM) compared to Sample C and Sample B (0.48-0.5 mM).

Under fed condition, pH decrease was higher with Sample C and Sample B (−0.6±0.01 Δ24-0 h), while lactate levels were significantly increased in both dual configurations (1.3-2.8 mM). Specifically, acetate and propionate were reduced in Sample C (acetate=35.0±0.8 mM; propionate=7.4±0.01) compared to the other conditions (acetate=38.2-42.5 mM; propionate=8-9.1 mM). Contrarily, the highest butyrate levels were detected in Sample B reactors (6.6±0.3 mM), and the opposite effect was observed for ammonium (108.8±4.2 mg/L). There were no significant differences in branched chain fatty acid production between the different capsules under fed conditions.

### Discussion of Results

Targeted delivery of pharmaceutically active compounds, nutritional supplements or probiotics is essential for providing the product performance and probiotic survivability and its function, including colonization and microbiome modulation.

The most common capsule material has been gelatin due to its accessibility, low price, non-toxicity, solubility in biological fluids at body temperature, and gelation characteristics. However, some disadvantages have been described for gelatin such as reactivity towards aldehyde groups, sugars, metal ions, plasticizers, or preservatives. In addition, moisture changes due to high environmental humidity, dependent temperature release, and animal (porcine) origin are all disadvantages of gelatin. HPMC fulfils multiple criteria for substituting gelatin-based capsules, as it is a plant-based material, has low cross-reactivity with excipients, is stable in a wide range of temperatures and moisture conditions and has a proven safety record for human consumption.

The aim of this research was to evaluate the release and disintegration characteristics of different HPMC-based capsule combinations in a capsule-in-capsule configuration, using caffeine bioavailability and probiotic survival as markers. SHIME model has been used to simulate the full length gastrointestinal tract conditions. We found that combinations which included Comp Sample C1 showed delayed caffeine release in the stomach and the small intestine under both fed and fasted conditions and confer a significant increase in probiotic viability and performance at the colonic level.

The nature and the concentration of the gelling agent dictate the release behavior. Our research showed that at the end of the fasted and fed gastric environment, caffeine release was complete in single Comp Sample C2 capsule while its profile was low with Comp Sample C1. Comp Sample C2 and Comp Sample C1 are both manufactured from HPMC, with gelling agent (gellan gum) incorporated in Comp Sample C1 compared to Comp Sample C2. Gellan insolubility at pH lower than 4 and changes in HPMC films physical properties with gelation, increased resistance to the mechanical stress during the gastric passage and may be responsible of the delayed release behavior of Comp Sample C1. It has been reported elsewhere that the HPMC capsules containing carrageenan as a gelling agent showed a fast disintegration profile in vivo under fasted conditions (complete release after 7-9 min), similar to gelatin capsules. In addition, gelling additives are also required for capsule shell HPMC manufacturing, because of the lower mechanical strength of the cellulosic film. Carrageenan and potassium chloride have been proven effective in HPMC gelation, while gellan gum combined with ethylene diamine tetraacetic acid (EDTA) or sodium citrate have been used in HPMC capsule production.

In the small intestinal phase, the highest delayed caffeine release was observed for Sample C under fasted conditions and for Comp Sample C1 under fed conditions, both not achieving, however, delivery of all the caffeine even at the end of the small intestine. This observation suggests that Sample C can be used for colonic-targeted delivery of viable probiotics at their site of action. Probiotic viability along with storage or administration are important factors of its efficacy. Thus, survival of orally administered probiotics is a requisite for their performance.

The caffeine release from Comp Sample C1 followed a linear trend (R2 >0.9) under both fed and fasted conditions, suggesting a steady-state delivery sustained in time, which may also be beneficial for probiotic engraftment in the gut. The change in the SCFA profile, suggest that other bacteria from the microbiota are affected by the introduction of the exogeneous L. acidophilus, indicating that this target delivery to the colon enabled modulation of the microbiome. In particular the observed increased in lactic acid, suggest conization by L. acidophilus. A viable “colonizer” microorganism in a sufficient mass, introduced in a complex ecosystem, can compete with other commensals thus modulating the diversity of the microbiome. This process is known as the propagule pressure hypothesis, where successful invasions require a sufficient number of individuals to enter the ecosystem, which relates to the cell numbers (or dose) of the treatment and frequency with which they are applied. Probiotic strains are not easily engrafting in the human gut ecosystem, due to the resilience of pre-established niches of commensal microorganisms. However, under dysbiotic conditions following antibiotic intake for example, the potential benefit of probiotic microorganisms to colonize and restore gut homeostasis may be improved by a targeted colonic delivery using capsule-in-capsule configuration. Indeed, previous research in vivo showed that acid resistant capsule in a capsule-in-capsule configuration were resistant to low pH gastric environment under fasted conditions. The same authors reported high interindividual variability in gastric emptying time, which can significantly affect disintegration times and product release. Despite in vivo conditions that may differ from in vitro tests due to the complex nature of the gastrointestinal processes and the inter-individual variability, different in vitro models simulating the gastrointestinal digestion have been developed to mimic the human physiology under fasted and fed conditions. Physiological gastric and intestinal pH and bile salts concentrations undergo gradual changes during the digestion processes, which were reproduced in this research by steady addition of acid and digestive fluids, improving the previously developed static settings (including duodenal, jejunal and ileal phases), with different pH, retention times, and bile salts concentrations, brought the in vitro systems closer the gastrointestinal digestion in humans.

Changes in caffeine release were accompanied by differences in viability of L. acidophilus, especially under fasted conditions. To further assess the function of L. acidophilus at its site of action, we evaluated if these changes in probiotic viability had an effect on gut microbial modulation under colonic conditions. Gastrointestinal digestion was continued with a simulated colonic fermentation for three selected capsules. Detection of viable L. acidophilus in the colonic environment was significantly higher when administered in Sample C or Sample B. Comp Sample C3 was used as a negative control, as suggested by lactic acid decrease. In addition, Sample C and Sample B also affected the microbial colonic composition and diversity based on the resulting decrease in acetate and propionate and increase in butyrate. Protection of L. acidophilus may have induced higher acidification of colonic media and lactate production, potentially by providing lactate as a substrate to other bacteria in the microbiota (cross-feeding interactions). It has been previously described that probiotic Lactobacillus spp. can ferment non-digestible fibers to enable lactate production, used subsequently as a substrate by butyrate-producing bacteria. Butyrate is a microbial metabolite with a key role in maintaining gut homeostasis, including immunoregulation, gut motility and epithelial barrier function. Increase in butyrate may reflects increase in butyrate-producing bacteria. Decrease in acetate and propionate may reflect a decrease in the se SCFA producing bacteria.

Low stomach pH and high bile acid concentrations are the major factors in reducing probiotic viability. Thus delayed-release formulations such as Comp Sample C1 or Sample B, targeting colonic delivery, may improve probiotic performance in modulating gut microbial diversity and composition, as observed in this study in vitro, leading to various health benefits. On the other hand, the fast caffeine release from Comp Sample C3 may suggest that this formulation can be used for targeted gastric release.

### Example 2

### MRI Test

Various capsule in capsule formulations were test for the effectiveness. The capsules and configurations are shown in Table 2

TABLE 2

| Study | | | |
|---|---|---|---|
| Arm | Outer capsule (size 00) | Inner capsule (size 3) | Observation time |
| I | HPMC capsule with | None | 60 min |
| | gelling agent | | |
| II | thermogelled HPMC | None | 60 min |
| | capsule | | |
| III | HPMC capsule with | HPMC capsule with | up to 90 min |
| | gelling agent | gelling agent | |
| IV | HPMC capsule with | thermogelled HPMC | up to 90 min |
| | gelling agent | capsule | |
| V | HPMC capsule with | acid resistant capsule | 180 min |
| | gelling agent | HPMC capsule | |
| VI | Gelatin capsule | acid resistant capsule | 180 min |
| | | HPMC capsule | |
| VII | acid resistant capsule | None | 180 min |
| | HPMC capsule | | |
| VIII | acid resistant capsule | HPMC capsule with | 180 min |
| | HPMC capsule | gelling agent | |
| IX | acid resistant capsule | thermogelled HPMC | 180 min |
| | HPMC capsule | capsule | |
| X | acid resistant capsule | acid resistant capsule | 240 min |
| | HPMC capsule | HPMC capsule | |

The capsules were filled with a powder mixture. The powder mixture used to fill the capsules is composed of common excipients that are known not to impact the capsules disintegration behavior. For the single capsules (study arms I, II and VII), the infill mixture consisted of 5% black iron oxide, 12% croscarmellose, 10%13C3 labelled caffeine (=25 mg) and standard capsule filling powder (99.5% mannitol and 0.5% silicon dioxide).

The capsule-in-capsule configurations (arms III to VI and VIII to X) were filled with a similar mixture, but amount of caffeine was greater on a percentage basis, since the inner capsules were smaller. It was decided that the caffeine content in the deliver forms would be constant at 25 mg in each capsule. The inner capsules mixture consisted of also 5% black iron oxide, 12% croscarmellose, 23%13C3-labelled caffeine (=25 mg) and standard capsule filling powder (99.5% mannitol and 0.5% silicon dioxide). The outer capsules infill was one of the smaller capsules (size 3, depending on the study arm) and the gap was filled with a mixture of 7.2% naturally occurring caffeine (=25 mg) and hibiscus tea powder. The amount of caffeine was about 50 mg per capsule (25 mg 13C3-labelled and 25 mg natural caffeine). All capsule types were filled by hand on a laboratory scale to target fill weight of 250 mg for single capsules size 00, 106 mg for capsules size 3 and 300 mg for capsule in capsule combination of size 00.

A healthy volunteer study was conducted. It was performed as open-label, single-center, 10-way cross-over study with at least 72 h wash out phase between the study days. 6 healthy young volunteers (2 male and 4 female) were recruited for this study. These included subjects had a mean age of 23.2±3.6 years and a mean BMI of 23.5±2.6 kg/m2. The volunteers were required to abstain caffeine-containing foods such as coffee, tea, and chocolate products for the duration of at least 3 days before and during each study day.

Each capsule type was investigated as a single capsule (arms I, II and VII). Furthermore, seven different capsule combinations were investigated. For each study arm, an observation time period was set based on the estimated maximum disintegration time of the individual capsules or the inner capsule of the capsule-in-capsule configurations shown in Table 2.

Between the study days were at least 72 h of wash out phase. All subjects arrived at the study unit in the morning after at least 10 h fast overnight. For each study arm, fasted MRI was obtained at −5 min and blank saliva probe was obtained at −2 min to ensure identical clinical conditions respectively. Time 0 min was defined as capsule intake in upright position together with 240 ml of water. All study arms consisted of 60 min of observation time with a 10 min interval, in study arms V to X additional 120 min observation time with a 15 min interval were added and in study arm X further 60 min observation time with a 15 min interval were performed. At each observation time point, two MRI sequences (TRUFI and VIBE) were applied in order to be able to distinguish between the two contrasting agents iron oxide and hibiscus tea powder. The sequence parameters are listed in Tables 3 and 4.

The T2*/T1 weighted TRUFI sequence is highly sensitive to the susceptibility artifact generated by magnetic materials like the applied ferrimagnetic black iron oxide. This characteristic artifact is independent of the hydration status and was therefore applied for the detection of the intact capsules. As soon as the capsule containing the iron oxide disintegrates, the iron oxide spreads which is visible as an enlargement of the artifact. In order to accelerate spreading of the powdered iron oxide the powerful disintegrant croscarmellose was added to the capsule filling mixture. In contrast, dry hibiscus tea powder is not visible in any sequence. However, as soon as it comes in contact with water the contained paramagnetic elements like manganese prolong the T1 water proton signal, which can be detected as a bright spot in the VIBE sequence. Therefore, the hibiscus tea powder label was aimed for the detection of the disintegration of the outer capsule. Nonetheless, the primary objective of the study was to investigate the fate of the inner capsule, which would be the relevant vehicle for clinical applications.

In case complete capsule disintegration could not be observed within the scheduled observation time, the measurement was extended by 30 min (two additional measurements). In case of the study arms with only a single capsule (I, II and VII), only the TRUFI sequence was performed, since in the single capsules no hibiscus tea powder was contained for which the VIBE sequence was aimed. Saliva samples were obtained always one minute after imaging.

MR imaging was performed using a Siemens MAGNETOM Aera MR-scanner (Siemens Healthcare, Erlangen, Germany) with a field strength of 1.5 Tesla in the Institute of Diagnostic Radiology and Neuroradiology in Greifswald. All measurements were performed in supine position (subject lying on the back, head forward). Two different spatial orientations (transversal and coronal) were used while the artifact was in the stomach, after gastric emptying only the coronal orientation was used.

TABLE 3
T2*/T1 weighted TRUFI sequence (iron oxide)

| | | |
|---|---|---|
| | Coronary | Transversal |
| Parameter | sequence settings | sequence settings |

| | | | | |
|---|---|---|---|---|
| Repetition time | 3.55 | ms | 3.55 | ms |
| Echo time | 1.48 | ms | 1.48 | ms |
| Slice thickness | 5.0 | mm | 5.0 | mm |
| Interslice gap | 0.75 | mm | 0.75 | mm |
| Voxel size | 3.04 | mm³ | 3.04 | mm³ |

| | | |
|---|---|---|
| Flipangle | 63° | 63° |

TABLE 4
T1 weighted VIBE (hibiscus tea powder)

| | | |
|---|---|---|
| | Coronary | Transversal |
| Parameter | sequence settings | sequence settings |

| | | | | |
|---|---|---|---|---|
| Repetition time | 3 | ms | 3 | ms |
| Echo time | 1.4 | ms | 1.4 | ms |
| Slice thickness | 3.0 | mm | 3.0 | mm |
| Interslice gap | 0 | mm | 0 | mm |
| Voxel size | 3.04 | mm³ | 3.04 | mm³ |

| | | |
|---|---|---|
| Flip angle | 30° | 30° |

### Image Analysis

Image analysis was performed using Horos Viewer Version 3.3.6. Tracking, assignment to the gastrointestinal compartments and evaluation of disintegration time points were performed manually. All recordings were independently evaluated by three independent observers, unclear findings were discussed.

The appearance of a bright spot in the VIBE sequence caused by wetted hibiscus tea powder was defined as the disintegration of the outer capsule. The time point of detected disintegration for the inner capsule in turn (detected in TRUFI sequence) was defined as the time of spreading of the characteristically shaped susceptibility artifact in the GI tract or visible sedimentation of the iron oxide within the stomach. The section of the GI-tract in which the artifact or corresponding particles (hibiscus tea powder or black iron oxide) were located at the time of the determined disintegration was assessed as the disintegration site of the respective (outer or inner) capsule.

### Results

The localization of the capsules and their disintegration could be clearly identified in the TRUFI sequences with exception of one administration of a Study Arm III combination and one administration of a Study Arm IV combination as disintegration took much longer than all other subjects in these study arms and thus longer than planned MRI observation time. Their disintegration could not be detected within the 30 additional minutes of imaging. The findings obtained by MRI are summarized in Tables 5 and 6. The results show that it is possible to delay the overall disintegration time with a capsule inside of another capsule. For some combinations, the disintegration time is almost exactly the sum of both disintegration times determined for the individual capsules e.g., 23 min for single Study Arm I and 40 min for Study Arm III. This was not the case when acid resistant capsules were included to the capsule-in-capsule configuration. Here the disintegration time of inner capsule was typically longer than the sum of disintegration times of outer and inner capsule. An increase in disintegration time was often accompanied with an increase in variability (±5 min and ±12 min for HPMC capsules with gelling agent and thermogelled HPMC compared to ±18 min for Study Arm IV).

The disintegration site of the single capsules as well as the capsule combinations were also quite variable (Table 5). Two exceptions were observed. The Study Arm II and the Study Arm X combination disintegrated most reproducible with their respective different properties. Thus, the combination of two acid resistant capsules resulted in all six administrations in a disintegration in the ileum. HPMC with a gelling agent and thermogelled HPMC and their combinations showed short disintegration times that resulted in disintegration within the stomach or proximal parts of the small intestine, whereas acid resistant capsules and their combinations with acid resistant capsule as outer shell mainly disintegrated in the small intestine. None of the tested combinations or single capsules reached the colon. Thermogelled HPMC capsules showed fast intragastric disintegration with very low variability in disintegration time and site. In total, 4 out of the 24 administrations with HPMC with a gelling agent capsules as outer shell and 1 out of the 24 administrations with acid resistant capsules as outer shell disintegrated in the esophagus. None of the subjects noticed any of the capsules adhering to the esophagus and none described any negative sensation.

TABLE 5
Gastrointestinal localization of disintegration
as determined by MRI

| Study Arm | Esophagus | Stomach | Duodenum | Jejunum | Ileum |
|---|---|---|---|---|---|
| I | 2 | 2 | 1 | 1 | — |
| II | — | 5 | — | 1 | — |
| VII | 1 | 1 | — | 4 | — |
| III | 1 | 3 | — | 2 | — |
| IV | — | 4 | — | 2 | — |
| V | 1 | 1 | 1 | 1 | 2 |
| VI | — | 4 | — | 1 | 1 |
| VIII | — | 1 | — | 4 | 1 |
| IX | — | 1 | — | 3 | 2 |
| X | — | — | — | — | 6 |

Results from Caffeine Determination

The results obtained by salivary caffeine determination are summarized together with MRI results in Table 6. The mean salivary 13C3-caffeine appearance times determined for the single size 00 capsules were 22±12 min for Study Arm I 15±0 min for Study Arm II and 25±11 min for Study Arm VII. These times are in good agreement with the salivary caffeine appearance times determined for the capsule-in-capsule configurations using these capsules as outer capsules. Consistent with the MRI results, the Study Arm X combination showed the longest salivary caffeine appearance time with 115±31 min and also the longest disintegration time with 123±25 min. And, also like observed with MRI, thermogelled HPMC capsules had the lowest variability in disintegration time as well as salivary caffeine appearance.

In Table 6 the mean disintegration times as determined by MRI and the salivary appearance times of natural caffeine and 13C3-labelled caffeine are shown. Furthermore, the time span between disintegration of inner and outer shell as well as gastric emptying time is given. In general, caffeine appears at the same time or earlier than the disintegration is detected by MRI. However, the trend towards later disintegration times and a higher variability for the capsule combinations is clear for both, and the ratios of the study arms are very similar to one another.

TABLE 6
Disintegration times of the capsules as determined by
salivary caffeine and MRI and gastric emptying times

| | | | | | | | Time span | | |
|---|---|---|---|---|---|---|---|---|---|
| | | | | | | | between | | |
| | Natural type | | | | | | appearance of | | |
| | caffeine | | Disintegration | | ¹³C₃- caffeine | | natural caffeine | | |
| | appearance | | time detected | | appearance | | from outer and | | |
| | time (outer | | by MRI (black | | time (inner | | ¹³C₃-caffeine | Gastric | |
| Study Arm | capsule) | | iron oxide) | | capsule) | | inner capsule | emptying time | |
| I | N/A | | 23 ± 12 | min | 22 ± 12 | min | N/A | 25 min | (n = 2) |
| II | N/A | | 12 ± 5 | min | 15 ± 0 | min | N/A | 15 min | (n = 1) |
| VII | N/A | | 37 ± 12 | min | 25 ± 11 | min | N/A | 15 ± 0 min | (n = 4) |
| III | 28 ± 9 | min | 40 ± 24 | min | 43 ± 20 | min | 11 ± 10 min | 30 min | (n = 2) |
| IV | 25 ± 8 | min | 40 ± 18 | min | 40 ± 23 | min | 16 ± 18 min | 20 min | (n = 2) |
| V | 22 ± 7 | min | 82 ± 38 | min | 57 ± 28 | min | 36 ± 24 min | 25 ± 12 min | (n = 4) |
| VI | 10 ± 5 | min | 74 ± 19 | min | 53 ± 17 | min | 43 ± 17 min | 35 min | (n = 2) |
| VIII | 28 ± 11 | min | 83 ± 33 | min | 65 ± 26 | min | 37 ± 23 min | 30 ± 20 min | (n = 5) |
| IX | 23 ± 12 | min | 75 ± 8 | min | 59 ± 16 | min | 41 ± 21 min | 44 ± 3 min | (n = 5) |
| X | 32 ± 7 | min | 123 ± 25 | min | 115 ± 31 | min | 83 ± 27 min | 41 ± 28 min | (n = 6) |

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A method of providing effective oral administration of an active substance to a mammal, where the active substance is delivered to the digestive tract of the mammal, said method comprising administering a delivery system to a mammal, the delivery system comprising:
an outer capsule having an outer shell wall and an internal chamber,
an inner capsule having an exterior shell wall and an inner compartment; the inner capsule being located in the internal chamber of the outer capsule, said inner capsule being acid resistant,
an active substance, wherein the active substance is present in the inner compartment of the inner capsule,
wherein the delivery system is orally administered to a mammal, and
wherein the delivery system delivers the active substance in an effective amount to the intestine of the mammal.

2. The method according to claim 1, wherein the outer capsule comprises an HPMC hard capsule.

3. The method according to claim 1, wherein the inner capsule comprises a HPMC hard capsule with acid resistance.

4. The method according to claim 3, wherein the inner capsule comprises a capsule comprising HPMC and gellan gum.

5. The method according to claim 4, wherein the gellan gum is present in an amount between about 4 parts-15 parts per 100 parts of the HPMC.

6. The method according to claim 2, wherein the HPMC capsule comprises thermogelled HPMC.

7. The method according to claim 1, wherein the outer capsule is an acid resistant capsule.

8. The method according to claim 7, wherein the outer capsule comprises a HPMC hard capsules with acid resistance.

9. The method according to claim 8, wherein the outer capsule comprises a capsule comprising HPMC and gellan gum.

10. The method according to claim 9, wherein the gellan gum is present in an amount between about 4 parts-15 parts per 100 parts of the HPMC.

11. The method according to claim 1, wherein the active substance comprises a probiotic.

12. The method according to claim 1, wherein the inner capsule and the outer capsule are each an acid resistant capsule and each acid resistant capsule comprises HPMC and gellan gum.

13. The method according to claim 12, wherein the gellan gum is present in an amount between about 4 parts-15 parts per 100 parts of the HPMC.

14. The method according to claim 1, wherein the inner capsule comprises more than one inner capsule.

15. The method according to claim 1, wherein the inner capsule is banned.

16. The method according to claim 1, wherein the active substance is delivered to the colon in an amount which is at least 10 times greater than a capsule which dissolves in the stomach or small intestine.

17. The method according to claim 16, wherein the active substance is delivered to the colon in an amount which is at least 20 times greater than a capsule which dissolves in the stomach or small intestine.

18. The method according to claim 17, wherein the active substance is delivered to the colon in an amount which is at least 30 times greater than a capsule which dissolves in the stomach or small intestine.

19. A method of modifying the microbiome and colonization of the gut by administering an active ingredient to the gut, said method comprising administering a delivery system to a mammal, the delivery system comprising:
an outer capsule having an outer shell wall and an internal chamber,
an inner capsule having an exterior shell wall and an inner compartment; the inner capsule being located in the internal chamber of the outer capsule, said inner capsule being acid resistant,
a probiotic active ingredient, wherein the active substance is present in the inner compartment of the inner capsule,
wherein the delivery system is orally administered to a mammal,
wherein the delivery system delivers the probiotic active substance in an effective amount to the gut of the mammal, and
wherein the active ingredient improves the microbiome or colonization of healthy bacteria in the gut.

20. The method according to claim 19, wherein outer capsule comprises an HPMC hard capsule.

21. The method according to claim 20, wherein the inner capsule comprises a HPMC hard capsules with acid resistance.

22. The method according to claim 20, wherein the inner capsule comprises a capsule comprising HPMC and gellan gum.

23. The method according to claim 22, wherein the gellan gum is present in an amount between about 4 parts-15 parts per 100 parts of the HPMC.

24. The method according to claim 20, wherein the HPMC capsule comprises thermogelled HPMC.

25. The method according to claim 19, wherein the outer capsule is an acid resistant capsule.

26. The method according to claim 25, wherein the outer capsule comprises a HPMC hard capsules with acid resistance.

27. The method according to claim 26, wherein the outer capsule comprises a capsule comprising HPMC and gellan gum.

28. The method according to claim 27, wherein the gellan gum is present in an amount between about 4 parts-15 parts per 100 parts of the HPMC.

29. The method according to claim 19, wherein the inner capsule and the outer capsule are each an acid resistant capsule and each acid resistant capsule comprises HPMC and gellan gum.

30. The method according to claim 29, wherein the gellan gum is present in an amount between about 4 parts-15 parts per 100 parts of the HPMC.

31. The method according to claim 19, wherein the inner capsule comprises more than one inner capsule

32. The method according to claim 19, wherein the inner capsule has a ban or banning substance at a junction of each part of the capsule.

33. The method according to claim 19, wherein the active substance is delivered to the colon in an amount which is at least 10 times greater than a capsule which dissolves in the stomach or small intestine

34. The method according to claim 33, wherein the active substance is delivered to the colon in an amount which is at least 20 times greater than a capsule which dissolves in the stomach or small intestine.

35. The method according to claim 34, wherein the active substance is delivered to the colon in an amount which is at least 30 times greater than a capsule which dissolves in the stomach or small intestine.
